SEQUENCE LISTING (5 sequences)

SEQ ID NO: 1 - DNA, 1701 bp, unidentified
  source 1..1701: mol_type = genomic DNA
atgaaggcaatactagtagttctgctgtatacatttacaaccgcaaatgcagacacattatgtataggttatcatgcgaacaattcaacagacactgtagacacagtactagaaaagaatgtaacagtaacacactctgtcaatcttctggaagacaagcataacggaaaactatgcaaactaagaggggtagccccattgcatttgggtaaatgtaacattgctggctggatcctgggaaatccagagtgtgaatcactctccacagcaagatcatggtcctacattgtggaaacatctaattcagacaatggaacgtgttacccaggagatttcatcaattatgaggagctaagagagcaattgagctcagtgtcatcatttgaaaggtttgaaatattccccaagacaagttcatggcctaatcatgactcggacaaaggtgtaacggcagcatgtcctcacgctggagcaaaaagcttctacaaaaacttgatatggctggttaaaaaaggaaattcatacccaaagctcaaccaaacctacattaatgataaagggaaagaagtcctcgtgctgtggggcattcaccatccacctactattgctgcccaagaaagtctctatcagaatgcagatgcatatgtttttgtggggacatcaagatacagcaagaagttcaagccggaaatagcaacaagacccaaagtgagggatcaagaagggagaatgaactattactggacactagtagaaccgggagacaaaataacattcgaagcaactggaaatctagtggtaccgagatatgcattcacaatggaaagagatgctggatctggtattatcatttcagatacaccagtccacgattgcaatacaacttgtcagacacccgagggtgctataaacaccagcctcccatttcagaatgtacatccgattacaattgggaaatgtccaaagtatgtaaaaagcacaaaattgagactggccacaggattgaggaatgtcccgtctattcaatctagaggcctattcggggccattgctggcttcattgaaggggggtggacagggatggtagatggatggtacggttatcaccatcaaaatgagcaggggtcaggatatgcagccgatctgaagagcacacaaaatgccattgataagattactaacaaagtaaattctgttattgaaaagatgaatacacagttcacagcagtgggtaaagagttcaaccaccttgaaaaaagaatagagaatctaaataaaaaagttgatgatggtttcctggacatttggacttacaatgccgaactgttggttctactggaaaacgaaagaactttggactatcacgattcaaatgtgaagaacttgtatgaaaaagtaagaaaccagttaaaaaacaatgccaaggaaattggaaacggctgctttgaattttaccacaaatgcgacaacacatgcatggaaagtgtcaagaatgggacttatgactacccaaaatactcagaggaagcaaaattaaacagagaaaaaatagatggagtaaagctggaatcaacaaggatctaccagattttggcgatctattcaactgttgccagttcattggtactggtagtctccctgggggcaatcagcttctggatgtgctctaatgggtctctacagtgtagaatatgtatttaa

SEQ ID NO: 2 - DNA, 1410 bp, unidentified
  source 1..1410: mol_type = genomic DNA
atgaatccaaaccaaaagataataaccattggctctatctgtatgacaattggaatggctaacttaatattacaaattggaaacataatctcaatatgggttagccactcaattcaaattggaaatcaaagccagattgaaacatgcaataaaaacgtcattacttatgaaaacaatacttgggtaaatcagacatatgttaacatcagcaacaccaactctgctgctagacagtcagtggcttccgtgaaattagcgggcaattcctctctctgccctgtcagtggatgggctatatacagtaaagacaacagtgtaagaatcggttccaagggggatgtgtttgtcataagggaaccattcatatcatgctctcccttggaatgcagaaccttcttcttgactcaaggggctttgctaaatgacaaacattccaatggaaccattaaagacagaagcccatatcgaaccctaatgagctgtcctattggtgaagttccctctccatacaactcaagatttgagtcagtcgcttggtcagcaagtgcttgtcatgatggcaccaattggctaacaattggaatttctggcccagacagtggggcagtggctgtgttaaaatacaatggcataataacagacactatcaagagttggaggaacaatatattgagaacacaagagtctgaatgtgcatgtgtaaatggttcttgctttaccataatgaccgatggaccaagtgatggacaggcctcatacaaaatcttcagaatagaaaagggaaagataatcaaatcagtcgaaatgaaagcccctaattatcactatgaggaatgctcctgttaccctgattctagtgaaatcacatgtgtgtgcagggataactggcatggctcgaatcgaccgtgggtgtctttcaaccagaatctggaataccagatgggatacatatgcagtggggttttcggagacaatccacgccctaatgataagacaggcagttgtggtccagtatcgtctaatggagcaaatggggtaaaaggattttcattcaaatacggcaatggtgtttggatagggagaactaagagcattagttcaagaaaaggttttgagatgatttgggatccgaatggatggactgggactgacaataaattctcaaaaaagcaagatatcgtaggaataaatgagtggtcagggtatagcgggagttttgttcagcatccagaactaacagggctgaattgtataagaccttgcttctgggttgaactaataagaggacgacccgaagagaacacaatctggactagcgggagcagcatatccttttgtggtgtagacagtgacattgtgggttggtcttggccagacggtgctgagttgccatttaccattgacaagtaa

SEQ ID NO: 3 - DNA, 1701 bp, unidentified
  source 1..1701: mol_type = genomic DNA
atgaagactatcattgctttgagctacattctatgtctggttttcactcaaaaaattcctggaaatgacaatagcacggcaacgctgtgccttgggcaccatgcagtaccaaacggaacgatagtgaaaacaatcacaaatgaccgaattgaagttactaatgctactgagttggttcagaattcctcaataggtgaaatatgcgacagtcctcatcagatccttgatggagggaactgcacactaatagatgctctattgggggaccctcagtgtgacggctttcaaaataagaaatgggacctttttgttgaacgaagcagagcctacagcaactgttacccttatgatgtgccggattatgcctcccttaggtcactagttgcctcatccggcacactggagtttaaaaatgaaagcttcaattgggctggagtcactcaaaacggaaaaagtttttcttgcataaggggatctagtagtagtttctttagtagattaaattggttgacccatttaaactacatatatccagcactgaacgtgactatgccaaacaaggaacaatttgacaaattgtacatttggggggttcaccatccggttacggacaaggaccaaatctccctgtatgctcaatcatcaggaagaatcacagtatctaccaaaagaagccaacaagctgtaatcccaaatatcggatctagacccagaataaggaatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtgaacaggatcacatacggggcctgtcccagatatgttaagcaaagcactctgaaattggcaacaggaatgcgaaatgtaccagagaaacaaactagaggcatatttggtgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctgaatcgattgatcggaaaaaccaacgagaaattccatcagattgaaaaagaattctcagaagtagaaggaagagttcaagaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggaaatggttgtttcaaaatataccacaaatgtgacaatgcctgcataggatcaataagaaatgaaacttatgaccacaatgtgtacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattagatgcaacatctgcatttga

SEQ ID NO: 4 - DNA, 1410 bp, unidentified
  source 1..1410: mol_type = genomic DNA
atgaatccaaatcaaaagataataacgattggctctgtttctctcacaatttccacaatatgcttcttcatgcaaattgccatcctgataactactgtaacattgcatttcaagcaatatgaattcaactcccccccgaataaccaagtgatgctgtgtgaaccaacaataatagaaagaaacataacagagatagtgtatttgaccaacaccaccatagagaaggaaatatgccccaaaccagcagaatacagaaattggtcaaaaccgcaatgtggcattacaggatttgcacctttctctaaggacaattcgattaggctttccgctggtggggacatctgggtgacaagagaaccttatgtgtcatgcgatcttgacaagtgttatcaatttgcacttggacagggaacaacactaaacaacgtgcattcaaataacacagtacgtgataggaccccttatcggactctattgatgaatgagttgggtgttcctttccatctggggaccaagcaagtgtgcatagcatggtccagctcaagttgtcacgatggaaaagcatggctgcatgtttgtataacaggggatgataaaaatgcaactgctagcttcatttacaatgggaggcttgtagatagtgttgtttcatggtccaacgatattctcaggacccaggagtcagaatgcgtttgtatcaatggaacttgtacagtagtaatgactgatggaaatgctacaggaaaagctgatactaaaatactattcattgaggaggggaaaatcgttcatactagcaaattgtcaggaagtgctcagcatgtcgaagagtgctcttgctatcctcgatatcctggtgtcagatgtgtctgcagagacaactggaaaggatccaaccggcccatcatagatataaacataaaggatcatagcattgtttccagttatgtgtgttcagggcttgttggagacacacccagaaaaagcgacagctccagcagtagccattgtttgaatcctaacaatgaagaaggtggtcatggagtgaaaggctgggcctttgatgatggaaatgacgtgtggatggggagaacaatcaatgagacgtcacgcttagggtatgaaaccttcaaagtcgttgaaggctggtccaaccctaagtccaaattgcagataaataggcaagtcatagttgacagaggtgataggtccggttattctggtattttctctgttgaaggcaaaagctgcatcaatcggtgcttttatgtggagttgattaggggaagaaaagaggaaactgaagtcttgtggacctcaaacagtattgttgtgttttgtggcacctcaggtacatatggaacaggctcatggcctgatggggcggacctcaatctcatgcatacataa

SEQ ID NO: 5 - DNA, 822 bp, unidentified
  source 1..822: mol_type = genomic DNA
atgaaattcttagtcaacgttgcccttgtttttatggtcgtgtacatttcttacatctatgcggacccgatcaacatgaccactagtatcaacaacaaccttcagcgtgtgcgtgaactggcggttcagtctgctaacagcgctgctgcaccaggagcagctgtcgacggtaccagccttctaaccgaggtcgaaacacctatcagaaacgaatgggggtccagatccaacgattcaagtgacgctgctgctggtggagcagctagccttctaaccgaggtcgaaacacctatcagaaacgaatgggggtccagatccaacgattcaagtgacgctgctgcaccaggagcagctagtcttctaaccgaggtcgaaacgcctaccagaagcgaatgggagtccagatccagcgattcaagtgatgctgctgctggtggagcagctagtcttctaaccgaggtcgaaacgcctaccagaaacgaatgggagtccagatccagcgattcaagtgatgctgctgcaccaggagcagctagtcttctaaccgaggtcgaaacgcttaccagaaacggatgggggtgcagatgcagcgattcaagtgatggtggactgaaacagattgaagataaattggaagagattttgagcaaactctatcatattgaaaacgaactggcgcgtattaaaaagctgctgggcgaactcgagattctggcgatctactcaactgtcgccagttcactggtgcttttggtctccctgggggcaatcagtttctggatgtgttctaatggatctttgcagtgcagaatatgcatctga

(19) United States
(12) Patent Application Publication
(10) Pub. No.: US 2026/0335425 A1
(43) Pub. Date: Oct. 29, 2026

(54) VIRUS-LIKE PARTICLE COMBINATION VACCINE COMPRISING INFLUENZA A VIRUS ANTIGEN PROTEIN HA, NA OR M2E5X

(71) Applicant: UNIVERSITY-INDUSTRY COOPERATION GROUP OF KYUNG HEE UNIVERSITY, Yongin-si (KR)

(72) Inventors: Bok Sil JEON, Seoul (KR); Keon Woong YOON, Yongin-si (KR)

(73) Assignee: UNIVERSITY-INDUSTRY COOPERATION GROUP OF KYUNG HEE UNIVERSITY, Yongin-si (KR)

(21) Appl. No.: 19/120,612

(22) PCT Filed: Sep. 26, 2023
(86) PCT No.: PCT/KR2023/014922

§ 371 Date: Apr. 11, 2025

(30) Foreign Application Priority Data
Oct. 11, 2022 (KR) 10-2022-0129531
Sep. 19, 2023 (KR) 10-2023-0124801

(51) Int. Cl. A61K 39/145; A61K 39/00; A61P 37/04; C12N 7/00

(52) U.S. Cl. CPC A61K 39/145; A61P 37/04; C12N 7/00; A61K 2039/5258; C12N 2760/16123; C12N 2760/16134; C12N 2760/16151

(57) ABSTRACT

The present invention relates to a virus-like particle combination vaccine comprising influenza A virus antigen protein HA, NA or M2e5x. A recombinant universal influenza-virus-like particle vaccine for protection against infection by influenza A virus (H1N1, H3N2 and H5N1) and influenza B virus (B/Victoria lineages) has been developed by preparing a virus-like particle vaccine expressed by a combination of: hemagglutinin (HA) and neuraminidase (NA) antigen proteins of 2020/2021 influenza viruses of A/Guangdong-Maonan/SWL1536/2019 (H1N1) and A/Hong Kong/2671/2019 (H3N2), which were recently predicted by the World Health Organization (WHO); and heterologous tandem M2e repeat (M2e5x) antigen proteins of conventional human, swine, and avian influenza viruses. The virus-like particle vaccine having high efficacy against various influenza, obtained by developing, on the basis of the present invention, a vaccine exhibiting high immunogenicity, can reduce infection and mortality caused by influenza A and B viruses during a global pandemic.

### TECHNICAL FIELD

The present disclosure relates to a virus-like particle combination vaccine including an influenza A virus antigen protein hemagglutinin (HA), neuraminidase (NA), or heterologous tandem M2e repeats (M2e5x).

### BACKGROUND ART

Influenza is an acute infectious disease that is highly contagious with symptoms such as fever, muscle pain, chills, and cough. Beginning in Mexico in 2009, influenza A viruses were confirmed in more than 214 countries, causing 18,500 deaths from April until the pandemic ended in August 2010. The leading candidates that may cause a current pandemic are avian influenza A (H5N1) and avian influenza A (H7N9) viruses (‘H5N1’ and ‘H7N9’). In the case of H5N1, 826 people were infected in 16 countries, and 440 of them died, showing a high mortality rate of about 53% (2015, WHO). Influenza B viruses account for 23% of all influenza infections and 52% of influenza-related pediatric deaths.

If an influenza pandemic were to occur, it is estimated that there would be around 7.4 million to 150 million deaths worldwide, causing the economic damage reaching $71 billion to $167 billion in the United States.

Currently, there is no report on the universal vaccine that can protect against both influenza A virus (A/PR/8/34, H1N1, A/Hong Kong/1/68, H3N2, A/Vietnam/2003, H5N1) and influenza B virus (B/Victoria lineages) infections, such that there is a great need for its development.

### DISCLOSURE OF THE INVENTION

### Technical Goals

An object of the present disclosure is to provide virus-like particles (VLPs) capable of inducing an immune response against an influenza virus expressing influenza A virus antigen proteins hemagglutinin (HA), neuraminidase (NA), or heterologous tandem M2e repeats (M2e5x) in combination.

In addition, another object of the present disclosure is to provide a vaccine composition including the virus-like particle as an active ingredient.

In addition, another object of the present disclosure is to provide an expression vector for preparing the virus-like particle, a host cell transformed by the expression vector, and a method of preparing the virus-like particle.

### Technical Solutions

To achieve the above objects, the present disclosure provides virus-like particles (VLPs) capable of inducing an immune response against an influenza virus, including hemagglutinin 3 (HA3), neuraminidase 1 (NA1), and neuraminidase 2 (NA2).

In addition, the present disclosure provides virus-like particles (VLPs) capable of inducing an immune response against an influenza virus, including hemagglutinin 1 (HA1) and hemagglutinin 3 (HA3).

In addition, the present disclosure provides virus-like particles (VLPs) capable of inducing an immune response against an influenza virus, including hemagglutinin 3 (HA3), neuraminidase 1 (NA1), and heterologous tandem M2e repeats (M2e5x).

In addition, the present disclosure provides virus-like particles (VLPs) capable of inducing an immune response against an influenza virus, including hemagglutinin 1 (HA1), hemagglutinin 3 (HA3), and heterologous tandem M2e repeats (M2e5x).

In addition, the present disclosure provides a vaccine composition including the virus-like particles as an active ingredient.

In addition, the present disclosure provides an expression vector for preparing a virus-like particle capable of inducing an immune response against an influenza virus, including a nucleic acid sequence which encodes HA3 and is represented by SEQ ID NO: 3; a nucleic acid sequence which encodes NA1 and is represented by SEQ ID NO: 2; and a nucleic acid sequence which encodes NA2 and is represented by SEQ ID NO: 4.

In addition, the present disclosure provides an expression vector for preparing a virus-like particle capable of inducing an immune response against an influenza virus, including a nucleic acid sequence which encodes HA1 and is represented by SEQ ID NO: 1 and a nucleic acid sequence which encodes HA3 and is represented by SEQ ID NO: 3.

In addition, the present disclosure provides an expression vector for preparing a virus-like particle capable of inducing an immune response against an influenza virus, including a nucleic acid sequence which encodes HA3 and is represented by SEQ ID NO: 3; a nucleic acid sequence which encodes NA1 and is represented by SEQ ID NO: 2; and a nucleic acid sequence which encodes M2e5x and is represented by SEQ ID NO: 5.

In addition, the present disclosure provides an expression vector for preparing a virus-like particle capable of inducing an immune response against an influenza virus, including a nucleic acid sequence which encodes HA1 and is represented by SEQ ID NO: 1; a nucleic acid sequence which encodes HA3 and is represented by SEQ ID NO: 3; and a nucleic acid sequence which encodes M2e5x and is represented by SEQ ID NO: 5.

Furthermore, the present disclosure provides a host cell transformed by the expression vector.

In addition, the present disclosure provides a method of preparing a virus-like particle capable of inducing an immune response against an influenza virus, including transforming a host cell with the expression vector; and culturing the host cell to express the virus-like particle.

### Advantageous Effects

The present disclosure relates to a virus-like particle combination vaccine including an influenza A virus antigen protein HA, NA, or M2e5x, wherein a universal recombinant influenza-virus-like particle vaccine that defends against infection by influenza A virus (H1N1, H3N2, and H5N1) and influenza B virus (B/Victoria lineages) has been developed by preparing a virus-like particle vaccine expressed by a combination of hemagglutinin (HA) and neuraminidase (NA) antigen proteins of 2020/2021 influenza viruses A/Guangdong-Maonan/SWL1536/2019 (H1N1) and A/Hong Kong/2671/2019 (H3N2), which were recently predicted by the World Health Organization (WHO), and heterologous tandem M2e repeat (M2e5x) antigen proteins of existing human, swine, and avian influenza viruses. With the development of the vaccine exhibiting high immunogenicity on the basis of the present disclosure, the virus-like particle vaccine having high efficacy against various influenza may reduce infection and mortality rate caused by influenza A and B viruses during a global pandemic.

### BRIEF DESCRIPTION OF DRAWINGS

FIG. 1 shows results of identifying characteristics of an influenza A virus-like particle.

FIG. 2 shows results of IgG influenza A and B specific antibody responses in serum.

FIG. 3 shows results of analyzing a virus titer in lungs after influenza A and B virus infection.

FIG. 4 shows results of analyzing rates of body weight changes and survival after influenza B virus infection.

FIG. 5 shows a plan for an influenza B virus animal experiment.

FIG. 6 shows results of a virus titer in lungs after influenza B virus infection.

FIG. 7 shows results of body weight changes and a survival rate after influenza B virus infection.

### BEST MODE FOR CARRYING OUT THE INVENTION

The present disclosure provides virus-like particles (VLPs) capable of inducing an immune response against an influenza virus, including hemagglutinin 3 (HA3), neuraminidase 1 (NA1), and neuraminidase 2 (NA2).

In addition, the present disclosure provides virus-like particles (VLPs) capable of inducing an immune response against an influenza virus, including hemagglutinin 1 (HA1) and hemagglutinin 3 (HA3).

In addition, the present disclosure provides virus-like particles (VLPs) capable of inducing an immune response against an influenza virus, including hemagglutinin 3 (HA3), neuraminidase 1 (NA1), and heterologous tandem M2e repeats (M2e5x).

In addition, the present disclosure provides virus-like particles (VLPs) capable of inducing an immune response against an influenza virus, including hemagglutinin 1 (HA1), hemagglutinin 3 (HA3), and heterologous tandem M2e repeats (M2e5x).

Preferably, the HA1 may be encoded by a nucleic acid sequence represented by SEQ ID NO: 1, the NA1 may be encoded by a nucleic acid sequence represented by SEQ ID NO: 2, the HA3 may be encoded by a nucleic acid sequence represented by SEQ ID NO: 3, the NA2 may be encoded by a nucleic acid sequence represented by SEQ ID NO: 4, and the M2e5x may be encoded by a nucleic acid sequence represented by SEQ ID NO: 5, but are not limited thereto.

Preferably, the influenza virus may be an influenza A virus or an influenza B virus, but is not limited thereto.

HA1 and NA1 used in the present disclosure are antigen proteins of A/Guangdong-Maonan/SWL1536/2019 (H1N1), wherein HA1 has Accession #: EPI1542570, and NA1 has Accession #: EPI1542569.

HA3 and NA2 used in the present disclosure are antigen proteins of A/Hong Kong/2671/2019 (H3N2), wherein HA3 has Accession #: EPI1698489, and NA2 has Accession #: EPI1698488.

As used herein, the term “virus-like particles (VLPs)” refer to non-infectious viral subunits that may or may not carry viral proteins. For example, the virus-like particles may completely lack a DNA or RNA genome or may undergo spontaneous self-assembly in the case of virus-like particles including viral capsid proteins.

As used herein, the term “virus-like particle capable of inducing an immune response against an influenza virus” refers to a protein structure (a particle) that induces a specific immune response against an influenza virus and has a virus-like shape.

The virus-like particle produces particles in a shape similar to a virus as structural proteins derived from the virus are assembled and includes an antigenic determining site derived from an influenza virus, thereby inducing a specific immune response against the influenza virus when the virus-like particle is injected into a specific individual. As a specific example of the present disclosure, the protein structure may have a form in which an antigenic determining site derived from an influenza virus is bound to the outside of a structural protein derived from the virus.

The virus-derived structural protein (core protein) may be influenza virus matrix protein 1 (M1).

As used herein, the term “influenza virus matrix protein 1 (M1)” is a structural protein of the influenza virus and refers to a matrix protein that forms a coat inside the lipid layer, which is an envelope of the influenza virus. The influenza virus includes eight fragmented negative-strand RNAs and proteins such as surface proteins hemagglutinin (HA), neuraminidase (NA), nucleoprotein (NP) matrix (M1), proton ion-channel protein (M2), polymerase acidic protein (PA) 1, polymerase basic protein 2 (PB2), polymerase acidic protein (PA), and nonstructural protein 2 (NS2), wherein the matrix protein 1 surrounds the outer shape in layers and acts as a linker between the core and the envelope. In the production of virus-like particles, the matrix protein 1 plays an important role in assembling the virus-like particles into a stable form.

The virus-like particle may act as an antigen in an organism and label antigens to T or B immune cells through reactions with antigen-labeled cells such as dendritic cells.

In addition, the present disclosure provides a vaccine composition including the virus-like particle as an active ingredient.

As used herein, the term “vaccine composition” refers to a composition that may prevent subsequent pathogen infection by administering a protein antigen having a vaccinating effect. The vaccine composition may be a composition including virus-like particles exhibiting antigenicity against an influenza virus.

Specifically, the vaccine composition may include any one selected from the group consisting of pharmaceutically acceptable carriers, adjuvants, immunostimulants, and a combination thereof.

The pharmaceutically acceptable carriers are known in the art and include proteins and sugars. The carrier may be an aqueous or non-aqueous solution, suspension, or emulsion. The non-aqueous carrier includes propylene glycol, polyethylene glycol, edible oils such as olive oil, and injectable organic esters such as ethyl oleate. The aqueous carrier includes water, alcohol/aqueous solutions, emulsions, or suspensions, including drinking water and buffer media. The parenteral carrier includes sodium chloride solution, Ringer's dextrose, dextrose and sodium chloride, lactated Ringer, or fixed oils. The intravenous carrier includes electrolyte supplements, liquids, and nutritional supplements, such as those based on Ringer's dextrose. Preservatives and other additives may further include those such as antimicrobial agents, antioxidants, chelating agents, and inert gases. The preservative may include, but is not limited to, formalin, thimerosal, neomycin, polymyxin B, and amphotericin B.

The vaccine composition may further include an adjuvant. The adjuvant refers to a compound or mixture that enhances the immune response and/or promotes the rate of absorption after vaccination, including any absorption-promoting agent. Acceptable adjuvants include, but are not limited to, Freund's complete preservative, Freund's incomplete preservative, saponins, mineral gels such as aluminum hydroxide, surfactants such as lysolecithin, pultron polyols, polyanions, peptides, oil or hydrocarbon emulsions, keyhole limpet hemocyanin, and dinitrophenol.

The vaccine composition may further include an immunostimulant. The immunostimulant may include artificially synthesized levamisole, isoprinosine, and cytokines. An example of cytokines includes interferon-α, interleukin-2, GM-CSF, and GCSF. The vaccine composition may be used in a form containing the virus-like particles or a concentrate thereof, or in the form of a dry powder of the transformed host cells themselves or the transformed cells. In addition, the vaccine composition may be used together with other foods or food ingredients and applied appropriately according to conventional methods.

The vaccine composition may further comprise one or more second adjuvants selected from the group consisting of stabilizers, emulsifiers, aluminum hydroxide, aluminum phosphate, pH adjusters, surfactants, liposomes, ISCOM adjuvants, synthetic glycopeptides, bulking agents, carboxypolymethylene, bacterial cell walls, derivatives of bacterial cell walls, bacterial vaccines, animal poxvirus proteins, subviral particle adjuvants, cholera toxin, N,N-dioctadecyl-N′,N′-bis(2-hydroxyethyl)-propanediamine, monophosphoryl lipid A, dimethyldioctadecyl-ammonium bromide, and a mixture thereof.

The vaccine composition may be used by being formulated in the form of oral formulations such as powder, granules, tablets, capsules, suspensions, emulsions, syrups, and aerosols, as well as sterile injection solutions according to conventional methods. When formulated, commonly used diluents or excipients such as fillers, bulking agents, binders, wetting agents, disintegrants, and surfactants may be used together.

Solid preparations for oral administration include tablets, pills, powders, granules, and capsules, and these solid preparations may be used, in a lecithin-like emulsifier, with at least one or more excipients such as starch, calcium carbonate, sucrose or lactose, and gelatin. In addition to the excipients, lubricants such as magnesium stearate and talc may also be used.

As the liquid preparations for oral administration, suspensions, solutions, emulsions, and syrups may be used, and in addition to commonly used simple diluents such as water and liquid paraffin, various excipients such as wetting agents, sweeteners, fragrances, and preservatives may be used.

Sterile aqueous solutions, non-aqueous preparations, suspensions, emulsions, and lyophilized preparations may be used for parenteral administration. For non-aqueous preparations and suspensions, propylene glycol, polyethylene glycol, vegetable oils such as olive oil, and injectable esters such as ethyl oleate may be used.

In addition, the present disclosure provides an expression vector for preparing a virus-like particle capable of inducing an immune response against an influenza virus, including a nucleic acid sequence which encodes HA3 and is represented by SEQ ID NO: 3; a nucleic acid sequence which encodes NA1 and is represented by SEQ ID NO: 2; and a nucleic acid sequence which encodes NA2 and is represented by SEQ ID NO: 4.

In addition, the present disclosure provides an expression vector for preparing a virus-like particle capable of inducing an immune response against an influenza virus, including a nucleic acid sequence which encodes HA1 and is represented by SEQ ID NO: 1 and a nucleic acid sequence which encodes HA3 and is represented by SEQ ID NO: 3.

In addition, the present disclosure provides an expression vector for preparing a virus-like particle capable of inducing an immune response against an influenza virus, including a nucleic acid sequence which encodes HA3 and is represented by SEQ ID NO: 3; a nucleic acid sequence which encodes NA1 and is represented by SEQ ID NO: 2; and a nucleic acid sequence which encodes M2e5x and is represented by SEQ ID NO: 5.

In addition, the present disclosure provides an expression vector for preparing a virus-like particle capable of inducing an immune response against an influenza virus, including a nucleic acid sequence which encodes HA1 and is represented by SEQ ID NO: 1; a nucleic acid sequence which encodes HA3 and is represented by SEQ ID NO: 3; and a nucleic acid sequence which encodes M2e5x and is represented by SEQ ID NO: 5.

The term “vector” as used herein refers to a nucleic acid molecule used to transport linked nucleic acid fragments. For the vector, a bacterium, a plasmid, a phage, a cosmid, an episome, a virus, and an insertable DNA fragment (i.e., a fragment insertable into the genome of host cells by homologous recombination) may be used, but are not limited thereto. The plasmid, a type of vector, refers to a circular double-stranded DNA loop into which an additional DNA fragment can be linked. Additionally, the viral vector is capable of linking additional DNA into a viral genome.

The term “expression vector” as used herein refers to a vector capable of directing the expression of a gene encoding an operably linked target protein. In general, expression vectors in the use of recombinant DNA technology are in the form of plasmids, and thus the terms plasmid and vector may be used interchangeably. However, it may also include other forms of expression vectors that perform the same function as viral vectors.

Specifically, the vector may be a baculovirus vector. The baculovirus as used herein refers to a pathogenic virus that is not pathogenic to humans or vertebrates but pathogenic only to insects.

The expression vector is introduced into a host cell, and the host cell transformed by the introduced vector may produce the virus-like particle. Here, the vector may include a promoter recognized by a host organism.

The nucleic acid sequence encoding the virus-like particle may be operably linked to the promoter sequence. The term “operably linked” as used herein refers to a state in which one nucleic acid fragment is combined with another nucleic acid fragment such that its function or expression is affected by the other nucleic acid fragment. That is, the gene encoding the virus-like particle may be operably linked to a promoter within the vector to regulate expression.

In addition, the expression vector may include an appropriate marker gene necessary for selecting transformed host cells. The marker gene may be an antibiotic-resistant gene or fluorescent protein gene, and the antibiotic-resistant gene may be selected from the group consisting of, but is not limited to, a hygromycin-resistant gene, a kanamycin-resistant gene, a chloramphenicol-resistant gene, and a tetracycline-resistant gene. The fluorescent protein gene may be selected from the group consisting of, but is not limited to, a gene of a yeast-enhanced green fluorescent protein (yEGFP), a gene of a green fluorescent protein (GFP), a gene of a blue fluorescent protein (BFP), and a gene of a red fluorescent protein (RFP).

Furthermore, the present disclosure provides a host cell transformed by the expression vector.

The term “host cell” as used herein refers to any organism that may be infected by a virus and immunized by virus-like particles. The host cells may be metabolically manipulated by transformation.

The host cell may be a microorganism, an animal cell, a plant cell, an animal-derived culture cell, or a plant-derived culture cell. The suitable host cell may be a naturally occurring or wild-type host cell or a modified host cell. The wild-type host cell may be a host cell that has not been genetically modified by recombination.

As used herein, the term “transformation” refers to a method of transporting the vector into a microorganism or a specific cell, and when the target cell for transformation is a prokaryotic cell, it may be carried out by a CaCl2 method, a Hanahan method, and electroporation. If the target cell for transformation is a eukaryotic cell, the method may be implemented via microinjection, calcium phosphate precipitation, electroporation, liposome-mediated transfection, DEAE-dextran treatment, and gene bombardment, but is not limited thereto. In the case of transformation of fungi such as yeast, it may generally be carried out by, but is not limited to, transformation using lithium acetate and heat shock as well as electroporation.

In addition, the present disclosure provides a method of preparing a virus-like particle capable of inducing an immune response against an influenza virus, including transforming a host cell with the expression vector; and culturing the host cell to express the virus-like particle.

The virus-like particle may be prepared by genetic engineering methods, and since it may be produced by genetic engineering methods without a separate causative infectant, i.e., the influenza virus in the present disclosure, high productivity and economic feasibility may be realized.

The virus-like particle may be prepared by methods well known in the art. For example, the virus-like particle may be produced by transforming a specific host cell with a recombinant DNA molecule encoding the structural protein and the antigen recognition site followed by culture, and the protein expressed within the cell may be assembled on the cell surface and then released to the culture supernatant. At this time, the surface protein included in the virus-like particle maintains its shape intact without undergoing an immobilization process, thereby inducing a desired immune response against a specific pathogen in an organism.

The transformed host cell may be cultured under batch, fed-batch, or continuous fermentation conditions, and the host cell may express the virus-like particle by transformation, enabling acquisition of the virus-like particle protein from the cultured host cell.

### MODES FOR CARRYING OUT THE INVENTION

Hereinafter, the present disclosure will be described in detail according to examples that do not limit the present disclosure. It should be noted that the following examples of the present disclosure are only intended to illustrate the present disclosure and do not limit or restrict the scope of the present disclosure. Therefore, what those skilled in the art to which the present disclosure pertains can easily infer from the detailed description and examples of the present disclosure is interpreted as falling within the scope of the present disclosure.

### <Example 1> Characterization Following Preparation of Influenza A Virus-Like Particle Vaccine Using HA1, NA1, HA3, NA2, and M2e5x Antigens of Influenza A Virus

HA and NA genes of an influenza A virus were cloned into a pFast vector. For recombinant baculovirus (rBV) expressing each antigen gene, recombinant plasmids were transfected into SF-9 cells using Cellfectine II reagent (Invitrogen Waltham MA USA) in a 12-well plate, followed by culture at 27 °C for 7 days. After checking that the sf-9 cells infected with baculovirus had grown in size under a microscope, they were cultured in 250 mL flasks with 2.5×10⁶ cells/mL antigens using 100 mL of sf-9 media at 27° C., 140 rpm. Each of the generated recombinant baculovirus (rBV) HA1, NA1, HA3, NA2, and M1 rBVs was centrifuged at 3000 rpm for 30 minutes to obtain rBVs. The prepared rBVs were cultured at 27° C., 140 rpm for 2-3 days at 2.5×10⁶ cells/mL in proportion to the VLP vaccine to be prepared followed by centrifugation at 6000 rpm for 30 minutes to obtain virus-like particle vaccines H3N1N2, H1H3, H3N1M2e, and H1H3M2e VLPs. The acquired VLPs were centrifugated at 30,000 rpm for one hour through 60%, 30%, and 15% sucrose density gradient and then the separated VLP layers were collected to obtain purified VLPs.

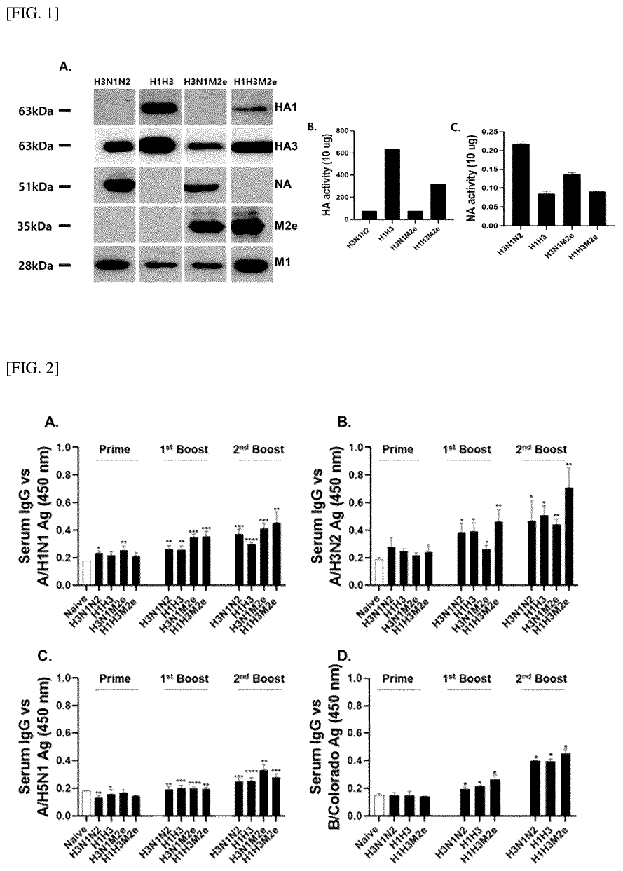

As shown in FIG. 1, it was found via Western blot that influenza A virus-like particles were produced with each of the antigen genes. The prepared virus-like particle vaccine was subjected to characterization for NA using monoclonal antibody and for HA using poly-clonal antibody. H3N1N2, H1H3, H3N1M2e, and H1H3M2e were successfully prepared.

### <Example 2> Verification of Vaccine Efficacy in Animal Models

Mice were vaccinated with various manufactured influenza A virus-like particle vaccines and infected with influenza A viruses (A/Puerto Rico/8/34, H1N1, A/HongKong/1/1968, H3N2, A/chicken/Vietnam/G04/2004, H5N1) and B/Colorado/06/2017 (Victoria lineage) so as to observe specific immune responses in the serum and lungs.

The manufactured H3N1N2, H1H3, H3N1M2e, and H1H3M2e VLPs were vaccinated intranasally to BALB/c mice three times at three-week intervals at a dose of 10 μg, serum was collected by mouse orbital blood sampling using heparin tubes at four-week intervals after vaccination, and the supernatant was obtained with a centrifuge at 5000 rpm for minutes after a reaction at room temperature for one hour. The serum obtained was coated with inactivated H1N1, H3N2, H5N1, and B/Colorado influenza viruses at 4 μg/ml each and then diluted (1:50), followed by a reaction at 37 °C for one hour. Afterwards, the degree of IgG 10 antibody formation that specifically reacts with influenza A and B viruses was analyzed via ELISA using HRP conjugated goat anti-mouse IgG (1:2000) as a secondary antibody.

As shown in FIG. 2, the groups of mice vaccinated with H3N1N2, H1H3, H3N1M2e, and H1H3M2e VLPs showed higher antibody responses than the Naive. As the number of vaccination increased, the antibody responses in the vaccine groups increased.

Despite the coating with influenza B virus antigen, an antibody response was derived upon vaccination with the VLP vaccine. Additionally, the M2e5x-containing vaccine-injected groups H3N1M2e and H1H3M2e VLPs showed higher influenza-specific antibody responses against all A and B influenza antigens.

### <Example 3> Identification of Virus Titers in Lungs Following Infection with Influenza A and B Viruses

After vaccination, each group of mice was infected intranasally with lethal doses of influenza A viruses {A/Puerto Rico/8/34 (H1N1) and A/HongKong/1/1968 (H3N2), A/chicken/Vietnam/G04/2004 (H5N1)} and B virus {B/Colorado/06/2017 (Victoria lineage)}. On the fourth day after infection, lung tissues were separated, ground, and centrifuged at 6000 rpm and 4° C., and then the supernatant was obtained. MDCK cells were cultured in 12-well plates at 37 °C until reaching a confluency of 100-110%. Afterwards, the virus supernatant obtained from the lung was subjected to plaque assay through 10-fold serial dilution. Culture was performed at 37 °C for approximately 3 to 4 days until plaques were formed. Afterwards, staining was performed with crystal violet.

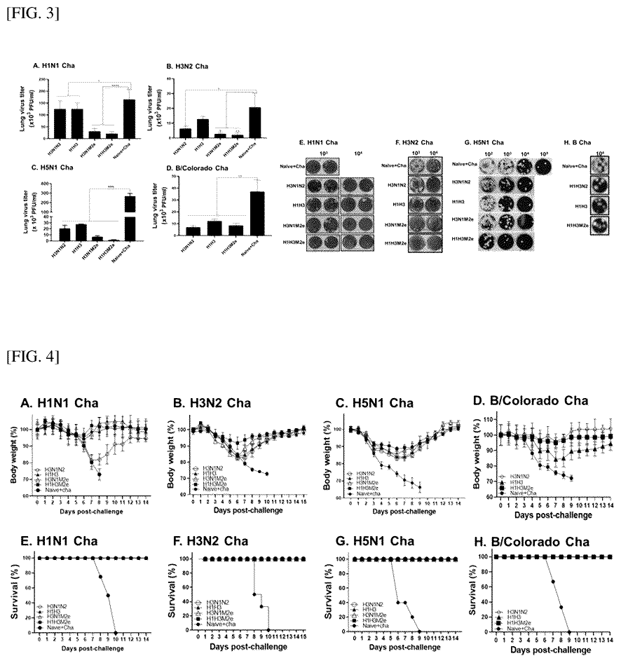

As shown in FIG. 3, the groups vaccinated with H3N1N2, H1H3, H3N1M2e, and H1H3M2e VLPs had lower virus titers compared to Naive+Cha which is the unvaccinated group. Although VLP vaccines were prepared using HA and NA derived from H1N1 and H3N2, significantly low virus titers were shown in H5N1, B/Victoria lineage compared to the Naive+Cha control group. In particular, for the M2e5x-containing vaccine-injected groups, the H3N1M2e and H1H3M2e VLP vaccines showed lower virus titers than the unvaccinated group for both influenza A viruses (H1N1, H3N2, H5N1) and influenza B viruses (B/Victoria lineage).

### <Example 4> Analysis of Rates of Body Weight Changes and Survival after Influenza B Virus Infection

Four weeks after the third vaccination, mice were infected with lethal doses of influenza A viruses {A/Puerto Rico/8/34 (H1N1) and A/HongKong/1/1968 (H3N2), A/chicken/Vietnam/G04/2004 (H5N1)} and influenza B virus {B/Colorado/06/2017 (Victoria lineage). The body weight and survival rate were observed every 24 hours for 14 days after infection.

As shown in FIG. 4, when infected by attack of the influenza A virus (H1N1, H3N2, H5N1), the H3N1N2, H1H3, and H3N1M2e VLP vaccinated groups showed a weight loss of approximately 15 to 20%. In particular, the H1H3M2e VLP vaccine showed the lowest weight loss rate at approximately 10%. All VLP vaccines showed a 100% survival rate.

When infected by attack of influenza B/Colorado/06/2017 (Victoria lineage) virus, the H1H3 VLPs vaccinated group showed a weight loss of about 20% or less on day 7 after attack, and in particular, the H3N1N2 and H1H3M2e VLP vaccines showed a weight loss of about 10%. All VLP vaccinated groups recovered on days 7 to 9, showing a 100% survival rate.

In conclusion, the influenza A virus-derived virus-like particle universal vaccines (H1H3M2e, H3N1M2e, H3N1N2, and H1H3 VLPs) prepared herein showed a 100% survival rate for infection with lethal doses of various influenza A viruses (A/Puerto Rico/8/34, H1N1, A/HongKong/1/1968, H3N2, A/chicken/Vietnam/G04/2004, H5N1), and the vaccine efficacy was in the order of H1H3M2e, H3N1M2e, H3N1N2, and H1H3 VLPs.

In particular, in the influenza B virus infection that has not yet been reported, all of the influenza A virus-derived H1H3M2e, H3N1N2, and H1H3 VLP vaccines showed a 100% survival rate, and the efficacy was in the order of H1H3M2e, H3N1N2, and H1H3 VLPs.

### <Example 5> Animal Infection Experiments with the Influenza B Virus

### 1. Experimental Method

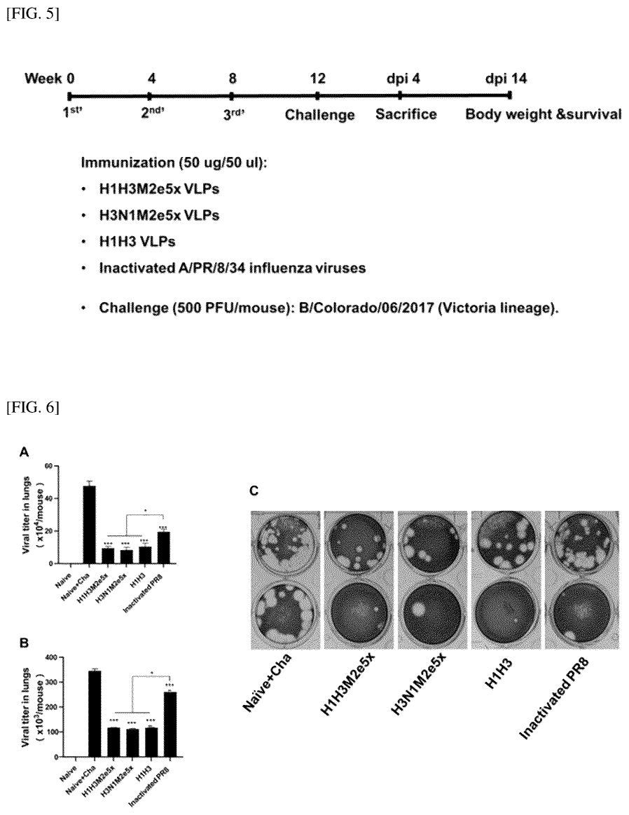

Virus-like particle vaccines expressing HA, NA, or M2e5X of an influenza A virus (H1H3M2e5x VLPs, H3N1M2e5x VLPs, H1H3 VLPs) were prepared, and inactivated influenza A virus (Inactivated A/PR/8/34) vaccine was prepared as a control. Each vaccine (50 μg/μl) was injected three times at 4-week intervals to mice (BALB/c) as suggested in the animal infection experiment plan. Four weeks after the third vaccination, the animals were infected with a lethal dose (500 pfu/ml) of influenza B virus (Colorado/06/2017, Victoria lineage). Mice were sacrificed on day 4 after infection, virus titers in lung tissues were measured, and body weights and survival rates were measured at the same time every day for 14 days after infection (FIG. 5).

### 2. Experimental Results

### (1) The Titer of Influenza B Virus in Lung Tissues

Mice vaccinated with VLP vaccines (H1H3M2e5x VLPs, H3N1M2e5x VLPs, H1H3 VLPs) had significantly reduced virus titers compared to unvaccinated normal controls. Additionally, the H1H3M2e5x, H3N1M2e5x, and H1H3 VLP vaccines showed virus titers that were approximately 2-2.5 times lower than those of the inactivated influenza A virus vaccine group (FIG. 6).

### (2) Examination of Mouse Body Weight Changes and Survival Rate

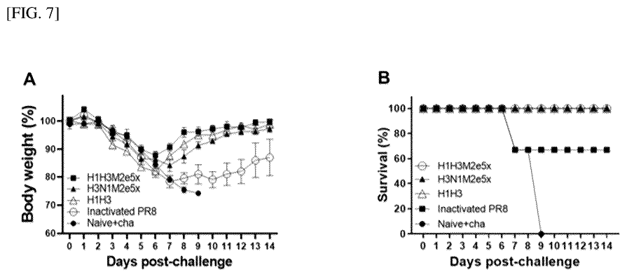

The virus-like particle vaccinated (H1H3M2e5x VLPs, H3N1M2e5x VLPs, H1H3 VLPs) groups showed similar body weight loss (15-20%), all recovered to normal body weight, and all survived. The control group vaccinated with inactivated influenza A virus did not recover to the normal body weight and showed a survival rate of approximately 60%. In contrast, all unvaccinated normal mice died. In conclusion, it was determined that the three types of VLP vaccines (H1H3M2e5x VLPs, H3N1M2e5x VLPs, H1H3 VLPs) have superior vaccine efficacy than the inactivated influenza A virus vaccine (FIG. 7).

While a specific part of the present disclosure has been described in detail above, it is clear for those skilled in the art that this specific description is merely preferred example embodiments, and the scope of the present disclosure is not limited thereby. Accordingly, the substantial scope of the present disclosure is defined by the attached claims and their equivalents.

1. (canceled)

2. Virus-like particles (VLPs) capable of inducing an immune response against an influenza virus, comprising hemagglutinin 1 (HA1) and hemagglutinin 3 (HA3).

3. (canceled)

4. (canceled)

5. The virus-like particles according to claim 2, wherein the HA1 is encoded by a nucleic acid sequence represented by SEQ ID NO: 1 and the HA3 is encoded by a nucleic acid sequence represented by SEQ ID NO: 3.

6. The virus-like particles according to claim 2, wherein the influenza virus is an influenza A virus or an influenza B virus.

7. A method of inducing an immune response against an influenza virus in a subject comprising administering a vaccine composition comprising Virus-like particles (VLPs) comprising hemagglutinin 1 (HA1) and hemagglutinin 3 (HA3) as an active ingredient to the subject.

8. The method of claim 7, wherein the HA1 is encoded by a nucleic acid sequence represented by SEQ ID NO: 1 and the HA3 is encoded by a nucleic acid sequence represented by SEQ ID NO: 3.

9. A method of preparing a virus-like particle comprising:
transforming a host cell with an expression vector for preparing the virus-like particle comprising a first nucleic acid sequence represented by SEQ ID NO: 1 encoding HA1 and a second nucleic acid sequence represented by SEQ ID NO: 3 encoding HA3; and
culturing the host cell to express the virus-like particle.

10-12. (canceled)

13. The method of claim 9, wherein the host cell is a microorganism, an animal cell, a plant cell, an animal-derived culture cell, or a plant-derived culture cell.

14. (canceled)
